(19) United States
(12) Patent Application Publication
(10) Pub. No.: US 2006/0270049 A1
(43) Pub. Date: Nov. 30, 2006

(54) Enhanced detection of analytes on surfaces using gold nanoparticles

(75) Inventor: William J. Todd, Baton Rouge, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(73) Assignee: Board of Supervisors of Louisiana State University & A & M college

(21) Appl. No.: 11/137,158

(22) Filed: May 25, 2005

(51) Int. Cl. G01N 33/00

(52) U.S. Cl. 436/80

(57) ABSTRACT

Adding free thiol compounds to colloidal gold markers enhances both the rate at which they bind to proteins or other analytes, and the sensitivity of those reactions. The addition of thiol is most effective when the thiol is added to the reaction mixture at about the same time as the gold nanoparticles. The presence of thiol compounds in colloidal gold staining reactions enhances both kinetics and thermodynamic equilibrium. The reaction time decreases, and the sensitivity of detection increases by approximately an order of magnitude.

This invention pertains to the detection of analytes, particularly biological molecules such as proteins, using gold nanoparticles.

Gold nanoparticles, particles of metallic gold whose diameters may range from a few nanometers to several hundred nanometers, display colors that range from orange to red to purple, with the color depending on particle size. Colloidal suspensions of gold nanoparticles may be prepared through means known in the art. These suspensions can be prepared with a relatively uniform distribution of particle sizes. The colors of gold nanoparticles are intense, meaning that they may readily be detected colorimetrically or by visual inspection even in low concentrations. For this reason, they are often used as labels in detection methods, to signal the presence or concentration of an analyte.

For example, colloidal gold has been used as a stain for proteins in qualitative and semi-quantitative Western blot analyses. Proteins are electrophoretically separated from one another by size, and the separated proteins are transferred from an electrophoresis gel to a nitrocellulose membrane or other solid support. See, e.g., B. D. Homes et al. (Eds.), Gel Electrophoresis of Proteins: a Practical Approach (1981); and Hoefer Scientific Instruments, Protein Electrophoresis Applications Guide (1994). The solid support with the separated proteins is then immersed in, or otherwise brought into contact with a colloidal gold reagent. See, e.g., M. Moeremans et al., “Sensitive colloidal metal (gold or silver) staining of protein blots on nitrocellulose membranes,” Anal. Biochem., vol. 145, pp. 315-321 (1985). Electrostatic interactions cause the gold nanoparticles to bind to the proteins, revealing not only the presence of the proteins, but also their approximate molecular weights, and estimated concentrations.

The literature contains numerous examples of the detection of proteins by colloidal gold staining in Western blots. See, e.g., A. Schapira, “Colloidal gold staining and immunodetection in 2D protein mapping, Meth. Mol. Biol., vol. 80, pp. 237-241 (1998); D. Egger et al., “Colloidal gold staining and immunoprobing on the same western blot,” Meth. Mol. Biol., vol. 80, pp. 217-222 (1998); and A. Schapira et al., “Two-dimensional protein mapping by gold stain and immunoblotting,” Anal. Biochem., vol. 169, pp. 167-171 (1988).

A similar approach may be used to estimate the amount of a sample .by applying aliquots of serially diluted sample onto a support, such as nitrocellulose, and then contacting the “dot blots” with a colloidal gold reagent. By comparing the intensities of the resulting colors to those for standard samples, the amount of protein in the original sample may be estimated.

Using these techniques, it is now routine to detect sub-nanogram amounts of protein in a single dot using colloidal gold reagents. However, the reaction rates are quite slow, generally requiring two to four hours or longer to achieve a sensitivity on the order of 0.01 nanogram. There have been occasional reports of sensitivity at the one picogram level, but only following overnight incubation.

Gold particles may be modified to alter their binding characteristics, for example by coating with positively charged molecules, to be used in detecting anions, or by coating with reagents such as protein A or an antibody to target specific compounds. See, e.g., M. Bendayan, “A Review of the Potential and Versatility of Colloidal Gold Cytochemical Labeling for Molecular Morphology,” Biotechnic & Histochemistry vol. 75, pp. 203-242 (2000).

Gold surfaces, including gold colloid surfaces, have been chemically modified, for example by using a sulfide/gold bond to link reagents to the surface. See M. Bendayan (2000); D. Marie-Christine et al., “Gold Nanoparticles: Assembly, Supramolecular Chemistry, Quantum-Size-Related Properties, and Applications toward Biology, Catalysis, and Nanotechnology,” Chem. Rev., vol. 104, pp. 293-346 (2004); and published international patent application WO 02/01228.

There is an unfilled need for a improved methods to detect picogram to nanogram levels of protein or other analytes with colloidal gold labels, methods that are specific, easy to implement, and rapid.

I have discovered that adding charged thiol compounds to colloidal gold nanoparticles enhances both the rate at which the nanoparticles bind to proteins or other analytes, and the sensitivity of the binding reaction. Interestingly, the addition of thiol is most effective when the thiol is added to the reaction mixture at about the same time as the gold nanoparticles—just prior to, simultaneously with, or shortly after contacting the protein with the nanoparticles. The presence of thiol compounds in colloidal gold staining reactions enhances both kinetics and thermodynamic equilibrium. The reaction time decreases substantially, and the sensitivity of detection increases by approximately an order of magnitude. In some examples, the reaction time decreases from about 4 hours to about 20 minutes. The higher sensitivity and faster reaction time both mean a less expensive analytical procedure. In practicing the present invention, there is no need to include an antibody or other high-affinity binding ligand in the reaction mixture. The thiol-containing compound is preferably a “free” thiol, meaning that the thiol group is preferably not covalently linked to a protein, peptide, or nucleic acid. Depending on the reaction conditions, gold particles bound to analyte are often observable within about 30 minutes, about 15 minutes, about 10 minutes, or even within about 5 minutes.

I have found that, surprisingly, not only can thiol-based compounds improve the binding of gold nanoparticles to protein surfaces, but also that they need not be prepared as part of the colloidal gold reagent. Adding free thiol compounds at the time when the gold particles react with the protein surface improves both the kinetics and the sensitivity of the labeling reaction. Adding the thiol too far in advance may be deleterious: over time the thiol-treated colloidal gold reagent loses its effectiveness, and the amount of background staining increases.

### EXAMPLES

Dot blot tests confirmed the effectiveness of thiols in improving both reaction rates and sensitivity of analyte detection. Stock reagents of colloidal gold were prepared by the method of J. Slot et al., “A new method of preparing gold probes for multiple-labeling cytochemistry,” J. Cell Biol., vol. 38, pp. 87-93 (1985). The prepared colloidal gold reagents were generally similar to commercially available colloidal gold reagents.

Proteins were first labeled with negatively-charged colloidal gold alone (i.e., without added thiols). The colloidal gold reagent was adjusted to pH 2.5 to pH 3.0 using a ten-fold dilution of concentrated hydrochloric acid. (Because colloidal gold solutions tend to ruin pH electrodes, approximate pH was determined using pH indicator paper.) This pH range was chosen to optimize interactions between negatively-charged colloidal gold particles and proteins, by choosing conditions favoring the formation of positively charged side groups on the protein. Bovine serum albumin (BSA) was used as the analyte protein in prototype testing. Nitrocellulose test strips (Schleicher & Schuell, pore size 0.45 μm) were prepared by dotting 1.0 μl aliquots of a series of ten-fold serial dilutions of BSA in phosphate buffered saline (PBS). The first dot contained 1 μg of BSA, and the last dot contained 0.01 μg. A dot with PBS only was used as a negative control. Additional tests were also conducted, beginning with 1 ng nanogram of BSA per dot, followed by ten-fold serial dilutions to successively lower concentrations of BSA.

Similar tests were then conducted, this time adding various negatively charged thiol molecules to the colloidal gold reagents. The total amount of gold in the solutions was 0.01% by weight, without further alterations in volume such as by dilution or concentration of the product. Two thiols that appeared to be particularly promising were thiolactic acid and thioglycolic acid. Thioglycolic acid, also known as mercaptoacetic acid, was selected for the next set of tests. Serial dilutions of thioglycolic acid (Sigma T-6750, 70% aqueous solution) showed that it was effective over a range of concentrations, at least from about 2 μL to about 24 μL or higher thioglycolate solution per 10 mL colloidal gold solution. However, at higher thioglycolic acid concentrations the mixed reagent was stable for only a few hours prior to use, and the background or noise level increased as well. Over time, the solution begins to stain hydrophobic groups in addition to the positively charged groups on the protein. Nitrocellulose itself is somewhat hydrophobic and begins to stain with an older solution, presenting background problems. It is unknown what reaction between the thiol and the gold particles (or other reaction) may account for this change over time. The gold colloid alone is stable in water for long periods of time.

BSA is commonly used as a model protein, or as a control in detection assays. Other proteins, peptides, or other analytes may be detected if the incubation conditions promote positive charges on the protein. Generally, the assays are run between about pH 2 and about pH 6. Alternatively, analytes with negative surface charges, generally at a pH about 5 or higher, may be detected by using thiol compounds with positively charged groups, e.g., amines.

A concentration of 8 μL of thioglycolic acid per 10 mL of colloidal gold was selected for comparison testing against other formulations of colloidal gold.

The thioglycolic acid-modified colloidal gold reagent was compared with both the unmodified colloidal gold reagent, and with three commercially-purchased colloidal gold reagents that are sold as stains for proteins on membrane supports. Otherwise identical test strips were prepared in a series of ten-fold dilutions, from 1.0 ng of BSA per dot down to 0.1 pg of BSA per dot. A dot made from the same volume of PBS carrier was included as a negative control in each test. At each concentration tested, the thioglycolic acid-treated colloidal gold reacted fastest. The faster reaction rates with the thiol were particularly noticeable at the lower protein concentrations, where positive results were observed after fifteen to twenty minutes with thiol, as compared to two hours for the colloidal gold reagents without thiol. After about 30 minutes, the thioglycolic acid-modified reagent routinely detected dots of 1 pg; and at an incubation time of 60 minutes it could detect protein samples down to about 0.1 pg of protein, which was the resolution limit in this set of experiments. There did not seem to be an appreciable increase in sensitivity when incubation time was increased beyond 60 minutes. Even after two to three hours, none of the other formulations unambiguously detected the 1 pg sample, although sensitivity at the 1 pg level following overnight incubation is claimed by the manufacturers. None of the other formulations successfully detected the 0.1 pg sample.

However, when the colloidal gold-thioglycolic acid mixture was stored for a time prior to use, its efficiency in staining protein decreased. Background staining of the nitrocellulose began to interfere with protein staining. The best results were obtained by adding the thiolglycolic acid to the colloidal gold and using the mixture within about 24 hours of mixing, preferably within about one hour of mixing. Surprisingly, when the reagent was used promptly, background staining of the nitrocellulose was actually less than was the case with an untreated gold reagent. It is unknown why this should be the case.

Without wishing to be bound by this theory, it is believed that the mechanism underlying the present invention is as follows. Native nanoparticles of gold remain suspended in the aqueous medium due to the mutual repulsion arising from their negative surface charges. The same negative surface charges attract the gold particles to positively-charged groups on the surface of the protein. For this reason the reactions are preferably conducted under acidic conditions, to favor the formation of positive charges on amino acid residues on the protein surface. The negative charge on the gold particles suffices both to maintain the particles in a suspended colloid indefinitely, and also to stain proteins more-or-less permanently. “Extending” the location of the charge further from the surface of the gold particles via the thiol linkage may help by reducing steric hindrance. Even at a diameter of 20 nm or so, the size of the gold particles generally used in Western Blots may encounter significant steric hindrance that blocks access to cationic sites on the protein surface.

It is quite surprising that the presence of thiols enhances the effectiveness of the gold nanoparticle labels. Conventional thinking would have suggested instead that the presence of excess or unbound ligands, such as negatively charged thiols, would interfere with the ability of the gold nanoparticles to bind by blocking access to the positively-charged binding sites on the protein, competitively inhibiting the ability of the gold marker to bind to the same sites. Surprising, this did not turn out to be the case.

Without wishing to be bound by this theory, I hypothesize that at least a part of the smaller thiol compound can better penetrate to interior portions of the protein, portions that might not be sterically accessible to the gold nanoparticles, while still leaving the thiol group projecting above the surface of the protein, accessible to react with the gold particles. However, even this rationale is incomplete: excess thiol compounds not bound to the protein would be present in substantial excess and would be expected to compete for binding sites on the gold particles, thereby blocking reaction of the gold particles with the exposed thiol bound to the protein. Thus it is quite surprising that the thiol compounds enhance binding of gold nanoparticles to the protein surface. It might be the case that binding affinity between the gold particles and thiol is significantly enhanced when the thiol compound is first attached to the protein, or vice versa; but why this should be is unknown. Data are not yet available to confirm whether this hypothesis is correct. Suggesting the contrary, on the other hand, was an observation that if the protein-dotted nitrocellulose test strips were preincubated in water containing thioglycolate at the same or a higher concentration, then rinsed briefly, and then exposed to standard, untreated colloidal gold reagent, the subsequent reaction with the dotted proteins was impeded.

Thus the mechanism underlying this invention remains uncertain, but it appears to proceed along lines that would not have been expected a priori. Indeed, the fact that it works at all is surprising. The superior results produced by the invention are still more surprising.

Just as negatively-charged thiol compounds may be used to target positively-charged amino acids on a protein surface, so may positively-charged thiol molecules be used to label anionic groups present on many biological molecules of interest —including more acidic proteins, carbohydrates, and nucleic acids. For example, I have observed that the thiol 2-mercaptoethylamine promoted binding of gold nanoparticles to bovine serum albumin at a pH of about 5 or higher, a pH that promotes the presence of negatively charged groups on the protein.

The complete disclosures of all references cited in this specification are hereby incorporated by reference. In the event of an otherwise irreconcilable conflict, however, the present specification shall control.

What is claimed:

1. A process for detecting an analyte on a surface; wherein the analyte contains one or more charged functional groups; wherein the presence of charge on the one or more functional groups may depend on pH; said process comprising the steps of:
(a) contacting the surface with an aqueous system; wherein the pH of the aqueous system promotes the formation of charge or the maintenance of charge on one or more of the functional groups of the analyte; wherein the aqueous system comprises a gold nanoparticle colloid; and wherein the aqueous system also comprises a solution of a compound that comprises both a thiol group and a group whose charge is opposite to the charge on the functional groups of the analyte at the pH of the aqueous system;
(b) allowing the surface to remain in contact with the aqueous system for a time sufficient for the thiol-containing compound to promote the fixing of gold nanoparticles onto the analyte; and
(c) observing any gold nanoparticles that have become fixed to the analyte.

2. A process as recited in claim 1, wherein said observing step is conducted visually.

3. A process as recited in claim 1, wherein said observing step is conducted by quantitative or semi-quantitative colorimetry, densitometry, visible spectroscopy, or visual inspection.

4. A process as recited in claim 1, wherein the gold nanoparticles are observable after being fixed to the analyte substantially more rapidly than would be the case in an otherwise identical process that lacked the thiol-containing compound.

5. A process as recited in claim 1, wherein the gold nanoparticles are observable after being fixed to the analyte; and wherein the concentration of analyte is sufficiently low that gold nanoparticles fixed to the analyte would not be observable in an otherwise identical process that lacked the thiol-containing compound, applying the same method of observation.

6. A process as recited in claim 1, wherein the thiol-containing compound is neither contacted with the analyte, nor mixed with the gold nanoparticles, sufficiently long before the gold nanoparticles are contacted with the analyte, to substantially impair the fixing of gold nanoparticles onto the analyte; as compared to an otherwise identical process in which the gold nanoparticles, the thiol-containing compound, and the analyte initially contact one another at substantially the same time.

7. A process as recited in claim 1, wherein one or more functional groups of the analyte have a positive charge at the pH of the aqueous system, and wherein the thiol-containing compound comprises a group that has a negative charge at the pH of the aqueous system.

8. A process as recited in claim 1, wherein one or more functional groups of the analyte have a negative charge at the pH of the aqueous system, and wherein the thiol-containing compound comprises a group that has a positive charge at the pH of the aqueous system.

9. A process as recited in claim 1, wherein the thiol-containing compound comprises thiolactic acid or thioglycolic acid.

10. A process as recited in claim 1, wherein the thiol-containing compound comprises 2-mercaptoethylamine.

11. A process as recited in claim 1, wherein the analyte is a protein.

12. A process as recited in claim 1, wherein the analyte is successfully detected in an amount less than about 10 pg.

13. A process as recited in claim 1, wherein the analyte is successfully detected in an amount less than about 5 pg.

14. A process as recited in claim 1, wherein the analyte is successfully detected in an amount less than about 1 pg.

15. A process as recited in claim 1, wherein the analyte is successfully detected in an amount less than about 0.5 pg.

16. A process as recited in claim 1, wherein the analyte is successfully detected in an amount of about 0.1 pg.

17. A process as recited in claim 1, wherein the gold nanoparticles are observable after being fixed to the analyte sooner than gold nanoparticles fixed to the analyte would be observable in an otherwise identical process that lacked the thiol-containing compound, applying the same method of observation.

18. A process as recited in claim 17, wherein the gold nanoparticles are observable after being fixed to the analyte within about 30 minutes.

19. A process as recited in claim 17, wherein the gold nanoparticles are observable after being fixed to the analyte within about 15 minutes.

20. A process as recited in claim 17, wherein the gold nanoparticles are observable after being fixed to the analyte within about 10 minutes.

21. A process as recited in claim 17, wherein the gold nanoparticles are observable after being fixed to the analyte within about 5 minutes.

22. A kit for detecting an analyte on a surface; wherein the analyte contains one or more charged functional groups; wherein the charge of a functional group may depend on pH; wherein said kit comprises:
(a) an aqueous gold nanoparticle colloid; and
(b) a compound that comprises both a thiol group and a group whose charge is complementary to the charge on the functional groups of the analyte at a selected pH; wherein the thiol group is not covalently linked to a protein, peptide, or nucleic acid;
wherein:
said aqueous gold nanoparticle colloid and said thiol-containing compound are packaged separately in said kit, so that said aqueous gold nanoparticle colloid and said thiol-containing compound do not mix with one another prematurely, before an end user causes them to be mixed.

23. A kit as recited in claim 22, wherein the thiol-containing compound comprises a group that has a negative charge at a selected pH.

24. A kit as recited in claim 22, wherein the thiol-containing compound comprises a group that has a positive charge at a selected pH.

25. A kit as recited in claim 22, wherein the thiol-containing compound comprises thiolactic acid or thioglycolic acid.

26. A kit as recited in claim 22, wherein the thiol-containing compound comprises 2-mercaptoethylamine.

27. A kit as recited in claim 22, wherein said kit does not contain an antibody or other high-affinity binding ligand.
